(19) United States
(12) Patent Application Publication
(10) Pub. No.: US 2004/0171576 A1
(43) Pub. Date: Sep. 2, 2004

(54) Adenosine a2a receptor agonist and an anticholinergic agent in combination for treating obstructive airways diseases

(76) Inventors: Michael Yeadon, Sandwich (GB); Roisin A Armstrong, Mystic, CT (US)

Correspondence Address: Robert T Ronau, Pfizer Inc, Patent Department Box 8260-1611, Eastern Point Road, Groton, CT, 06340, US

(21) Appl. No.: 10/479,085

(22) PCT Filed: May 24, 2002
(86) PCT No.: PCT/EP02/05725

Related U.S. Application Data
(60) Provisional application No. 60/293,842, filed on May 25, 2001.

(30) Foreign Application Priority Data
Dec. 6, 2001 (GB) 0129275.4
May 3, 2002 (GB) 0210238.2

(51) Int. Cl. A61K 31/7076; A61K 31/4745; A61L 9/04; A61K 9/14

(52) U.S. Cl. 514/45; 514/291; 424/46

(57) ABSTRACT

The present invention relates to a combination of a selective adenosine A₂ₐ receptor agonist and an anticholinergic agent for simultaneous, sequential or separate administration by the inhaled route in the treatment of an obstructive airways or other inflammatory disease, with the proviso that the anticholinergic agent is not a tiotropium salt.

The present invention relates to an inhaled combination of a selective adenosine A₂ₐ receptor agonist and an anticholinergic agent, with the proviso that the anticholinergic agent is not a tiotropium salt. The invention further relates to pharmaceutical compositions, including devices for administering, and to the uses of such a combination.

A combination of a selective adenosine A₂ₐ receptor agonist and an anticholinergic agent is useful in the treatment of obstructive airways and other inflammatory diseases, particularly the obstructive airways diseases asthma, chronic obstructive pulmonary disease (COPD) and other obstructive airways diseases exacerbated by heightened bronchial reflexes, inflammation, bronchial hyper-reactivity and bronchospasm. The combination is especially useful in the treatment of COPD.

Examples of particular diseases that may be treated with the present invention include the respiratory diseases asthma, acute respiratory distress syndrome, chronic pulmonary inflammatory disease, bronchitis, chronic bronchitis, chronic obstructive pulmonary (airway) disease and silicosis and diseases of the immune system such as allergic rhinitis and chronic sinusitis.

Adenosine has a wide range of physiologic activities, including immune and inflammatory responses, which are receptor-mediated and involve interaction with at least four types of plasma membrane receptors. These receptors are commonly referred to as A₁, A₂ₐ, A_{2b}, and A₃. Adenosine and its analogs have been found to possess a broad spectrum of anti-inflammatory activity that involves a significant variety of immune and inflammatory cells, including neutrophils and eosinophils. Activation of the A₂ₐ receptors on neutrophils results in the suppression of the production of reactive oxidants and other mediators of inflammation such as elastase by these cells, as well as decreased expression of β₂-integrins.

A₂ₐ receptors are known to exist on lymphocytes, neutrophils, eosinophils, basophils, monocytes/macrophages, epithelial cells, and on the vascular endothelial tissue with which they interact. Adenosine binding to A₂ₐ receptors can decrease inflammation by influencing the activities of a number of these cell types. For example, A₂ₐ receptor agonists markedly inhibit oxidative species elicited by physiologic stimulants such as neutrophil chemoattractants, cytokines, and lipid products.

Occupancy of adenosine A₂ₐ receptors stimulates neutrophil adenylyl cyclase, which results in an increase in intracellular cyclic AMP. In turn, increased neutrophil cyclic AMP results in depression of stimulated-neutrophil oxidative activity. Through a related action on a variety of other inflammatory cell types, the anti-inflammatory properties of A₂ₐ agonists extends beyond inhibitory activities on neutrophils. Adenosine also decreases endotoxin-stimulated monocyte/macrophage TNFa release, and it has been observed that endogenous adenosine as well as adenosine analogs reduce human monocyte TNFα production by binding to adenosine A₂ₐ receptors.

Endotoxin-stimulated release of interleukin-6 (IL-6) and interleukin-8 (IL-8) is decreased by adenosine analogs with an order of potency that suggests A₂ₐ adenosine receptor activity. Interleukin-10 (IL-10) has anti-inflammatory activity as a result of its ability to decrease endotoxin-stimulated TNFα release from monocytes, to inhibit oxidative activity, and to lower the expression of leukocyte adhesion molecules. Adenosine enhances stimulated human monocyte production of IL-10; consequently, the binding of adenosine at A₂ₐ receptors promotes resolution of any on-going inflammatory response that may be involved.

Activated eosinophils transmigrate into tissues and cause cellular damage and inflammation in such diseases as allergic and non-allergic asthma, allergic rhinitis, and atopic dermatitis. Adenosine and adenosine A₂ₐ receptor agonist analogs, by binding to A₂ₐ receptors on eosinophils, inhibit stimulated release of reactive oxygen species, a response which parallels the inhibitory effect of A₂ₐ receptors on neutrophils.

Further, inhaled A₂ₐ agonists inhibit the recruitment of eosinophils into lungs of sensitised guinea-pigs via action in the lungs (see WO-A-99/67263). This is important as A₂ₐ agonists relax blood vessels and lower blood pressure in animals thus the anti-inflammatory action of A₂ₐ agonists is ideally produced by an inhaled agent which has a high therapeutic index for activity in the lung compared with the peripheral compartment.

Anticholinergic agents prevent the effects resulting from passage of impulses through the parasympathetic nerves. This action results from their ability to inhibit the action of the neurotransmitter acetylcholine by blocking its binding to muscarinic cholinergic receptors. There are at least three types of muscarinic receptor subtypes. M₁ receptors are found primarily in brain and other tissue of the central nervous system, M₂ receptors are found in heart and other cardiovascular tissue and M₃ receptors are found in smooth muscle and glandular tissues. The muscarinic receptors are located at neuroeffector sites on, e.g., smooth muscle, and in particular M₃-muscarinic receptors are located in airway smooth muscle. Consequently, anti-cholinergic agents may also be referred to as muscarinic receptor antagonists.

The parasympathetic nervous system plays a major role in regulating bronchomotor tone, and bronchoconstriction is largely the result of reflex increases in parasympathetic activity caused in turn by a diverse set of stimuli. Anti-cholinergic agents have a long history of use in the treatment of chronic airway diseases characterised by partially reversible airway narrowing such as COPD and asthma and were used as bronchodilators before the advent of epinephrine. They were thereafter supplanted by β-adrenergic agents and methylxanthines. However, the more recent introduction of ipratropium bromide has led to a revival in the use of anti-cholinergic therapy in the treatment of respiratory diseases. There are muscarinic receptors on peripheral organ systems such as salivary glands and gut and therefore the use of systemically active muscarinic receptor antagonists is limited by side-effects such as dry mouth and constipation. Thus the bronchodilatory and other beneficial actions of muscarinic receptor antagonists is ideally produced by an inhaled agent which has a high therapeutic index for activity in the lung compared with the peripheral compartment.

Anti-cholinergic agents also partially antagonize bronchoconstriction induced by histamine, bradykinin, or prostaglandin F_{2α}, which is deemed to reflect the participation of parasympathetic efferents in the bronchial reflexes elicited by these agents.

It has now been surprisingly found that a combination of a selective adenosine A₂ₐ receptor agonist and an anticholinergic agent offers significant benefits in the treatment of obstructive airways and other inflammatory diseases over treatment with either agent alone. The advantage of the combination is to provide optimal control of airway calibre through the mechanism most appropriate to the disease pathology, namely muscarinic receptor antagonism, together with effective suppression of inappropriate inflammation. By combining both antimuscarinic and A₂ₐ agonist compounds via the inhaled route, the benefits of each class are realised without the unwanted peripheral effects. Further, the combination results in unexpected synergy, producing greater efficacy than maximally tolerated doses of either class of agent used alone.

The invention therefore provides an inhaled combination of a selective adenosine A₂ₐ receptor agonist and an anticholinergic agent, with the proviso that the anticholinergic agent is not a tiotropium salt.

Further, the invention provides an inhaled combination of a selective adenosine A₂ₐ receptor agonist and an anticholinergic agent for use as a medicament, with the proviso that the anticholinergic agent is not a tiotropium salt.

Further, the invention provides a combination of a selective adenosine A₂ₐ receptor agonist and an anticholinergic agent for simultaneous, sequential or separate administration by the inhaled route in the treatment of an obstructive airways or other inflammatory disease, with the proviso that the anticholinergic agent is not a tiotropium salt.

Further, the invention provides a pharmaceutical composition comprising a selective adenosine A₂ₐ receptor agonist, an anticholinergic agent and a pharmaceutically acceptable excipient, diluent or carrier, for administration by the inhaled route in the treatment of an obstructive airways or other inflammatory disease, with the proviso that the anticholinergic agent is not a tiotropium salt.

Further, the invention provides the use of a selective adenosine A₂ₐ receptor agonist or an anticholinergic agent in the manufacture of a medicament for simultaneous, sequential or separate administration of both agents by the inhaled route in the treatment of an obstructive airways or other inflammatory disease, with the proviso that the anticholinergic agent is not a tiotropium salt.

Further, the invention provides a method of treating of an obstructive airways or other inflammatory disease comprising administering simultaneously, sequentially or separately, by the inhaled route, to a mammal in need of such treatment, an effective amount of a selective adenosine A₂ₐ receptor agonist and an anticholinergic agent, with the proviso that the anticholinergic agent is not a tiotropium salt.

Further, the invention provides an inhalation device for simultaneous, sequential or separate administration of a selective adenosine A₂ₐ receptor agonist and an anticholinergic agent in the treatment of an obstructive airways or other inflammatory disease, with the proviso that the anticholinergic agent is not a tiotropium salt.

A selective adensoine A₂ₐ receptor agonist has a greater affinity for the adenosine A₂ₐ receptor than all other known adenosine receptors. Preferably, the affinity of such a selective adensoine A₂ₐ receptor agonist is at least 100 fold greater for the adensoine A₂ₐ receptor as compared with its affinity for the other adenosine receptors.

Suitable selective adenosine A₂ₐ-receptor agonists for use in the invention include the compounds generally and specifically disclosed in WO-A-00/23457, WO-A-00/77018, WO-A-01/27131, WO-A-01/27130, WO-A-01/60835, WO-A-02/00676 and WO-A-01/94368.

WO-A-00/23457 discloses a compound of the formula (I)

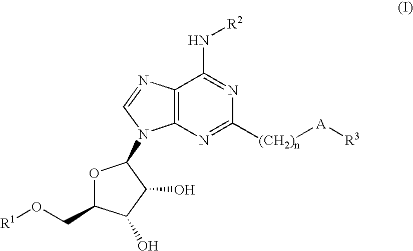

wherein

R¹ is alkyl or cyclopropylmethyl;

R² is phenyl-alkylene or naphthyl-alkylene, said alkylene chain being optionally further substituted by phenyl or naphthyl, each phenyl or naphthyl being optionally substituted by one or more substituents each independently selected from alkyl, alkoxy, halo and cyano;

n is 1 or 2;

A is NR^{a}, NR^{a}C(O), NR^{a}C(O)NR^{a}, NR^{a}C(O)O, OC(O)NR^{a}, C(O)NR^{a}, NR^{a}SO₂, SO₂NR^{a}, O, S or SO₂;

R^{a} is H, alkyl or benzyl optionally ring-substituted by one or more substituents each independently selected from alkyl, alkoxy, halo and cyano;

R³ is a group of the formula —(CH₂)ₚ—R^{p}—B;

p is 0, 1 or 2;

R^{p} is a bond, alkylene, cycloalkylene, phenylene or naphthylene, said cycloalkylene, phenylene and naphthylene each being optionally substituted by one or more substituents each independently selected from alkyl, alkoxy, halo and alkoxyalkylene;

B is

(i) H, —NR^{b}R^{b}, R^{b}R^{b}N-alkylene, —OR^{b}, —COOR^{b}, —OCOR^{b}, —SO₂R^{b}, —CN,

(ii) —SO₂NR^{b}R^{b}, —NR^{b}COR^{b}, —NR^{b}SO₂R^{b} or —CONR^{b}R^{b}, in which each R^{b} is the same or different and is selected from H, alkyl, phenyl and benzyl, provided that,

(a) when B is —OCOR^{b}, —SO₂R^{b}, —NR^{b}COR^{b} or —NR^{b}SO₂R^{b}, then the terminal R^{b} is not H, and,

(b) R^{p} is a bond, p is 0 and B is H only when A is NR^{a}, NR^{a}C(O)NR^{a}, OC(O)NR^{a}, C(O)NR^{a}, SO₂NR^{a}, O or S,

(ii) an optionally-substituted, fully- or partially-saturated or -unsaturated, mono- or bicyclic, heterocyclic group, which is linked to R^{p} by a ring carbon atom, or

(iii) N-linked azetidinyl, pyrrolidinyl, piperidinyl, piperazinyl or morpholinyl, each optionally substituted by one or more alkyl substituents, with the proviso that —(CH₂)ₚ—R^{p}— is not —CH₂—; and

where A is NR^{a}, C(O)NR^{a}, OC(O)NR^{a} or SO₂NR^{a}, R^{a} and R³ taken together with the nitrogen atom to which they are attached can form an azetidine, pyrrolidine, piperidine or piperazine ring, optionally substituted by one or more alkyl substituents:

and pharmaceutically acceptable salts and solvates thereof.

In a second aspect WO-A-00/23457 discloses a compound of the formula (I), as shown above, wherein

R¹ is C₁-C₆ alkyl or cyclopropylmethyl;

R² is phenyl-(C₁-C₆)-alkylene or naphthyl-(C₁-C₆)-alkylene, said C₁-C₆ alkylene chain being optionally further substituted by phenyl or naphthyl, each phenyl or naphthyl being optionally substituted by one or more substituents each independently selected from C₁-C₆ alkyl, C₁-C₆ alkoxy, halo and cyano;

n is 1 or 2;

A is NR^{a}, NR^{a}C(O), NR^{a}C(O)NR^{a}, NR^{a}C(O)O, OC(O)NR^{a}, C(O)NR^{a}, NR^{a}SO₂, SO₂NR^{a}, O, S or SO₂;

R^{a} is H, C₁-C₆ alkyl or benzyl optionally ring-substituted by one or more substituents each independently selected from C₁-C₆ alkyl, C₁-C₆ alkoxy, halo and cyano;

R³ is a group of the formula —(CH₂)ₚ—R^{P}—B;

p is 0, 1 or 2;

R^{p} is a bond, C₁-C₆ alkylene, C₃-C₇ cycloalkylene, phenylene or naphthylene, said C₃-C₇ cycloalkylene, phenylene and naphthylene each being optionally substituted by one or more substituents each independently selected from C₁-C₆ alkyl, C₁-C₆ alkoxy, halo and C₁-C₆ alkoxy-C₁-C₆-alkylene;

B is

(i) H, —NR^{b}R^{b}, R^{b}R^{b}N—(C₁-C₆)-alkylene, —OR^{b}, —COOR^{b}, —OCOR^{b}, —SO₂R^{b},

(ii) —CN, —SO₂NR^{b}R^{b}, —NR^{b}COR^{b}, —NR^{b}SO₂R^{b} or —CONR^{b}R^{b}, in which each R^{b} is the same or different and is selected from H, C₁-C₆ alkyl, phenyl and benzyl, provided that,

(a) when B is —OCOR^{b}, —SO₂R^{b}, —NR^{b}COR^{b} or —NR^{b}SO₂R^{b}, then the terminal R^{b} is not H, and,

(b) R^{p} is a bond, p is 0 and B is H only when A is NR^{a}, NR^{a}C(O)NR^{a}, OC(O)NR^{a}, C(O)NR^{a}, SO₂NR^{a}, O or S,

(i) an optionally-substituted, fully- or partially-saturated or -unsaturated, mono- or bicyclic, heterocyclic group, which is linked to R^{p} by a ring carbon atom, or

(ii) N-linked azetidinyl, pyrrolidinyl, piperidinyl, piperazinyl or morpholinyl, each optionally substituted by one or more C₁-C₆ alkyl substituents, with the proviso that —(CH₂)ₚ—R^{P}— is not —CH₂—; and

where A is NR^{a}, C(O)NR^{a}, OC(O)NR^{a} or SO₂NR^{a}, R^{a} and R³ taken together with the nitrogen atom to which they are attached can form an azetidine, pyrrolidine, piperidine or piperazine ring, each optionally substituted by one or more C₁-C₆ alkyl substituents:

and pharmaceutically acceptable salts and solvates thereof.

In a third aspect WO-A-00/23457 discloses a compound of the formula (I), as shown above, wherein

R¹ is alkyl or cyclopropylmethyl;

R² is phenyl-alkylene or naphthyl-alkylene where the alkylene chain may be substituted with methyl, ethyl, phenyl or naphthyl;

n is 1 or 2; and

A is NR^{a}, NR^{a}C(O), NR^{a}C(O)NR^{a}, NR^{a}C(O)O, OC(O)NR^{a}, C(O)NR^{a}, NR^{a}SO₂, SO₂NR^{a}, O, S or SO₂, in which R^{a} is H or alkyl;

R³ is a group of the formula —(CH₂)ₚ—R^{P}—B, wherein p is 0, 1 or 2;

R^{p} is a bond, or is alkylene, optionally alkyl-substituted cycloalkylene, phenylene or naphthylene; and

B is (i) H, —NR^{b}R^{b}, —OR^{b}, —COOR^{b}, —OCOR^{b}, —SO₂R^{b}, —CN, —SO₂NR^{b}R^{b}, —NR^{b}COR^{b}

or —CONR^{b}R^{b}, in which each R^{b} is the same or different and is selected from H and alkyl, provided that, (a) when B is —SO₂R^{b} or —NR^{b}COR^{b}, then the terminal R^{b} is other than H, and, (b) R^{p} is a bond, p is 0 and B is H only when A is NR^{a}, NR^{a}C(O)NR^{a}, C(O)NR^{a}, SO₂NR^{a}, O or S, or (ii) B is an optionally-substituted, fully or partially saturated or unsaturated mono- or bicyclic heterocyclic group, each of which is linked through a ring carbon atom;

or a pharmaceutically acceptable salt and solvate thereof.

WO-A-00/77018 discloses a compound of the formula:

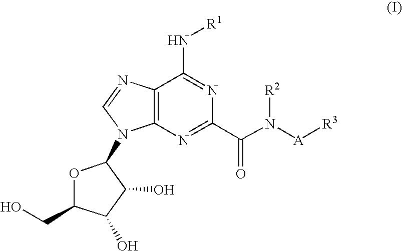

or a pharmaceutically acceptable salt or solvate thereof, wherein

R¹ is hydrogen or C₁-C₆alkyl optionally substituted by 1 or 2 substituents each independently selected from phenyl and naphthyl, said phenyl and naphthyl being optionally substituted by C₁-C₆ alkyl, C₁-C₆ alkoxy, halo or cyano;

R² is H or C₁-C₆ alkyl;

A is C₁-C₆ alkylene;

R³ is (i) hydrogen, C₁-C₆ alkyl, —COOR⁴, —CN, —CONR⁴R⁴ , C₃-C₈ cycloalkyl, phenyl or naphthyl, said C₃-C₈ cycloalkyl, phenyl and naphthyl being optionally substituted by C₁-C₆ alkyl, phenyl, C₁-C₆ alkoxy(C₁-C₆)alkyl, R⁴R⁴N(C₁-C₆)alkyl, halo(C₁-C₆)alkyl, fluoro(C₁-C₆)alkoxy, C₂-C₅ alkanoyl, halo, —OR⁴, cyano, —COOR⁴, C₃-C₈ cycloalkyl, —S(O)ₘR⁵, —NR⁴R⁴, —SO₂NR⁴R⁴, —CONR⁴R⁴, —NR⁴COR⁵ or —NR⁴SO₂R⁵,

or (ii) when A is C₂-C₆ alkylene, —NR⁴R⁴, —OR⁴, —OCOR⁵, —SO₂R⁵, —SO₂NR⁴R⁴ or —NR⁴COR⁵,

or (iii) a C-linked, 4- to 11-membered ring, mono- or bicyclic, heterocycle having either from 1 to 4 ring nitrogen atom(s), or 1 or 2 nitrogen and 1 oxygen or 1 sulphur ring atoms, being optionally C-substituted by oxo, C₁-C₆ alkoxy(C₁-C₆)alkyl, R⁶R⁶N(C₁-C₆)alkyl, halo(C₁-C₆)alkyl, fluoro(C₁-C₆)alkoxy, fluoro(C₂-C₅)alkanoyl, halo, cyano, —OR⁶, R⁷, —COR⁶, —NR⁶R⁶, —COOR⁶, —S(O)ₘR⁷, —SO₂NR⁶R⁶, —CONR⁶R⁶, —NR⁶SO₂R⁷ or —NR⁶COR⁷ and optionally N-substituted by C₁-C₆ alkoxy(C₁-C₆)alkyl, R⁶R⁶N(C₂-C₆)alkyl, halo(C₁-C₆)alkyl, fluoro(C₂-C₅)alkanoyl, R⁷, —COR⁶, —COOR⁷, —SO₂R⁷, —SO₂NR⁶R⁶ or —CONR⁶R⁶,

or (iv) when A is C₂-C₆ alkylene, N-linked azetidinyl, pyrrolidinyl, piperidinyl, piperazinyl, homopiperazinyl or morpholinyl, each being optionally C-substituted by C₁-C₆ alkyl, phenyl, C₁-C₆ alkoxy(C₁-C₆)alkyl, R⁴R⁴N(C₁-C₆)alkyl, halo(C₁-C₆)alkyl, fluoro(C₁-C₆)alkoxy, C₂-C₅ alkanoyl, halo, —OR⁴, cyano, —COOR⁴, C₃-C₈ cycloalkyl, —S(O)ₘR⁵, —NR⁴R⁴, —SO₂NR⁴R⁴, —CONR⁴R⁴, —NR⁴COR⁵ or —NR⁴SO₂R⁵, and said piperazinyl and homopiperazinyl being optionally N-substituted by C₁-C₆ alkyl, phenyl, C₁-C₆ alkoxy(C₂-C₆)alkyl, R⁴R⁴N(C₂-C₆)alkyl, fluoro(C₁-C₆)alkyl, C₂-₅ alkanoyl, —COOR⁵, C₃-C₈ cycloalkyl, —SO₂R⁵, —SO₂NR⁴R⁴ or —CONR⁴R⁴;

R⁴ is H, C₁-C₆ alkyl, C₃-C₈ cycloalkyl or phenyl;

R⁵ is C₁-C₆ alkyl, C₃-C₈ cycloalkyl or phenyl;

R⁶ is H, C₁-C₆ alkyl, C₃-C₈ cycloalkyl, phenyl, naphthyl or het;

R⁷ is C₁-C₆ alkyl, C₃-C₈ cycloalkyl, phenyl, naphthyl or het;

m is 0, 1 or 2; and

“het”, used in the definitions of R⁶ and R⁷, means C-linked pyrrolyl, imidazolyl, triazolyl, thienyl, furyl, thiazolyl, oxazolyl, thiadiazolyl, oxadiazolyl, pyridinyl, pyrimidinyl, pyridazinyl, pyrazinyl, indolyl, isoindolyl, quinolinyl, isoquinolinyl, benzimidazolyl, quinazolinyl, phthalazinyl, benzoxazolyl or quinoxalinyl, each being optionally substituted by C₁-C₆ alkyl, C₁-C₆ alkoxy, cyano or halo.

WO-A-01/27131 discloses a compound of the formula

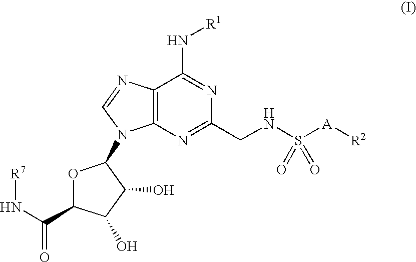

or a pharmaceutically acceptable salt or solvate thereof, wherein

R¹ is hydrogen or C₁-C₆ alkyl optionally substituted by 1 or 2 substituents each independently selected from phenyl and naphthyl, said phenyl and naphthyl being optionally substituted by C₁-C₆ alkyl, C₁-C₆ alkoxy, halo or cyano;

A is a bond or C₁-C₃ alkylene;

R² is (i) hydrogen, C₁-C₆ alkyl, C₃-C₇ cycloalkyl, phenyl or naphthyl, said C₃-C₇ cycloalkyl, phenyl or naphthyl being optionally substituted by C₁-C₆ alkyl, phenyl, C₁-C₆ alkoxy-(C₁-C₆)-alkyl, R³R³N-(C₁-C₆)-alkyl, fluoro-(C₁-C₆)-alkyl, fluoro-(C₁-C₆)-alkoxy, C₂-C₅ alkanoyl, halo, —OR³, cyano, —COOR³, C₃-C₇ cycloalkyl, —S(O)ₘR⁴, —NR³R³, —SO₂NR³R³ —CONR³R³, —NR³COR⁴ or —NR³SO₂R⁴, with the proviso that R² is not hydrogen when A is a bond,

or (ii) when A is C₂-C₃ alkylene, —NR⁸R⁹, —OR³, —COOR³, —OCOR⁴, —SO₂R⁴, —CN, —SO₂NR³R³, —NR³COR⁴or —CONR³R³,

or (iii) a C-linked, 4 to 11 membered, mono or bicyclic heterocycle having either from 1 to 4 ring nitrogen atom(s) or 1 or 2 nitrogen and 1 oxygen or 1 sulphur ring atoms, optionally C-substituted by oxo, C₁-C₆ alkoxy-(C₁-C₆)-alkyl, R³R³N—(C₁-C₆)-alkyl, fluoro-(C₁-C₆)-alkyl, fluoro-(C₁-C₆)-alkoxy, fluoro-(C₂-C₅)-alkanoyl, halo, cyano, —OR⁵, R⁶, —COR⁵, —NR⁵R⁵, —COOR⁵, —S(O)ₘR⁶, —SO₂NR⁵R⁵, —CONR⁵R⁵, —NR⁵SO₂R⁶ or —NR⁵COR⁶ and optionally N-substituted by C₁-C₆ alkoxy-(C₁-C₆)-alkyl, R³R³N—(C₂-C₆-alkyl, fluoro-(C₁-C₆)-alkyl, fluoro-(C₂-C₅)-alkanoyl, R⁶, —COR⁵, —COOR⁵, —S(O)ₘR⁶, —SO₂NR⁵R⁵ or —CONR⁵R⁵;

R³ is H, C₁-C₆ alkyl, C₃-C₇ cycloalkyl or phenyl;

R⁴ is C₁-C₆ alkyl, C₃-C₇ cycloalkyl or phenyl;

R⁵ is H, C₁-C₆ alkyl, C₃-C₇ cycloalkyl, phenyl, naphthyl or het;

R⁶ is C₁-C₆ alkyl, C₃-C₇ cycloalkyl, phenyl, naphthyl or het;

m is 0, 1 or 2;

“het”, used in the definitions of R⁵ and R⁶, means C-linked pyrrolyl, imidazolyl, triazolyl, thienyl, furyl, thiazolyl, oxazolyl, thiadiazolyl, oxadiazolyl, pyridinyl, pyrimidinyl, pyridazinyl, pyrazinyl, quinolinyl, isoquinolinyl, benzimidazolyl, quinazolinyl, phthalazinyl, benzoxazolyl or quinoxalinyl, each optionally substituted by C₁-C₆ alkyl, C₁-C₆ alkoxy, cyano or halo;

R⁷ is methyl, ethyl or cyclopropylmethyl; and

either, R⁸ and R⁹, taken together with the nitrogen atom to which they are attached represent azetidinyl, pyrrolidinyl, piperidinyl, morpholinyl, piperazinyl, homopiperidinyl, homopiperazinyl or tetrahydroisoquinolinyl, each being optionally substituted on a ring carbon atom by C₁-C₆ alkyl, C₃-C₈ cycloalkyl, phenyl, C₁-C₆ alkoxy-(C₁-C₆)-alkyl, R³R³N—(C₁-C₆)-alkyl, fluoro-(C₁-C₆)-alkyl, —CONR³R³, —COOR³ or C₂-C₅ alkanoyl, and optionally substituted on a ring carbon atom not adjacent to a ring nitrogen atom by fluoro-(C₁-C₆)-alkoxy, halo, —OR³, cyano, —S(O)ₘR⁴, —NR³R³, —SO₂NR³R³, —NR³COR⁴ or —NR³SO₂R⁴, and said piperazin-1-yl and homopiperazin-1-yl being optionally substituted on the ring nitrogen atom not attached to A by C₁-C₆ alkyl, phenyl, C₁-C₆ alkoxy-(C₂-C₆)-alkyl, R³R³N—(C₂-C₆)-alkyl, fluoro-(C₁-C₆)-alkyl, C₂-C₅ alkanoyl, —COOR⁴, C₃-C₈cycloalkyl, —SO₂R⁴, —SO₂NR³R³ or —CONR³R³,

or, R⁸ is H, C₁-C₆ alkyl, C₃-C₈ cycloalkyl, phenyl or benzyl and R⁹ is H, C₁-C₆ alkyl, C₃-C₈ cycloalkyl, phenyl, benzyl, fluoro-(C₁-C₆)-alkyl, —CONR³R³, —COOR⁴, C₂-C₅ alkanoyl or —SO₂NR³R³.

WO-A-01/27130 discloses a compound of the formula

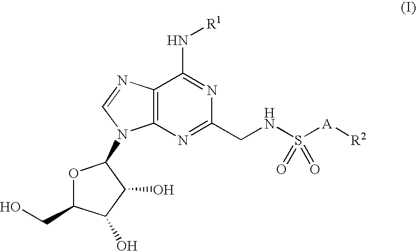

or a pharmaceutically acceptable salt or solvate thereof, wherein

R¹ is hydrogen or C₁-C₆ alkyl optionally substituted by 1 or 2 substituents each independently selected from phenyl and naphthyl, said phenyl and naphthyl being optionally substituted by C₁-C₆ alkyl, C₁-C₆ alkoxy, halo or cyano;

A is a bond or C₁-C₃ alkylene; p1 R² is (i) hydrogen, C₁-C₆ alkyl, C₃-C₇ cycloalkyl, phenyl or naphthyl, said C₃-C₇ cycloalkyl, phenyl or naphthyl being optionally substituted by C₁-C₆ alkyl, phenyl, C₁-C₆ alkoxy-(C₁-C₆)-alkyl, R³R³N—(C₁-C₆)-alkyl, fluoro-(C₁-C₆)-alkyl, fluoro-(C₁-C₆)-alkoxy, C₂-C₅ alkanoyl, halo, —OR³, cyano, —COOR³, C₃-C₇ cycloalkyl, —S(O)ₘR⁴, —NR³R³, —SO₂NR³R³, —CONR³R³, —NR³COR⁴ or —NR³SO₂R⁴, with the proviso that R² is not hydrogen when A is a bond,

or (ii) when A is C₂-C₃ alkylene, —NR⁷R⁸, —OR³, —COOR³, —OCOR⁴, —SO₂R⁴, —CN, —SO₂NR³R³, —NR³COR⁴ or —CONR³R³,

or (iii) a C-linked, 4 to 11 membered, mono or bicyclic heterocycle having either from 1 to 4 ring nitrogen atom(s) or 1 or 2 nitrogen and 1 oxygen or 1 sulphur ring atoms, optionally C-substituted by oxo, C₁-C₆ alkoxy-(C₁-C₆)-alkyl, R³R³ N—(C₁-C₆)-alkyl, fluoro-(C₁-C₆)-alkyl, fluoro-(C₁-C₆)-alkoxy, fluoro-(C₂-C₅)-alkanoyl, halo, cyano, —OR⁵, R⁶, —COR⁵, —NR⁵R⁵, —COOR⁵, —S(O)ₘR⁶, —SO₂NR⁵R⁵, —CONR⁵R⁵, —NR⁵SO₂R⁶ or —NR⁵COR⁶ and optionally N-substituted by C₁-C₆ alkoxy-(C₁-C₆)-alkyl, R³R³N—(C₂-C₆)-alkyl, fluoro-(C₁-C₆)-alkyl, fluoro-(C₂-C₅)-alkanoyl, R⁶, —COR⁵, —COOR⁵, —S(O)ₘR⁶, —SO₂NR⁵R⁵or —CONR⁵R⁵;

R³ is H, C₁-C₆ alkyl, C₃-C₇ cycloalkyl or phenyl;

R⁴ is C₁-C₆ alkyl, C₃-C₇ cycloalkyl or phenyl;

R⁵ is H, C₁-C₆ alkyl, C₃-C₇ cycloalkyl, phenyl, naphthyl or het;

R⁶ is C₁-C₆ alkyl, C₃-C₇ cycloalkyl, phenyl, naphthyl or het;

either, R⁷ and R⁸, taken together with the nitrogen atom to which they are attached represent azetidinyl, pyrrolidinyl, piperidinyl, morpholinyl, piperazinyl, homopiperidinyl, homopiperazinyl or tetrahydroisoquinolinyl, each being optionally substituted on a ring carbon atom by C₁-C₆ alkyl, C₃-C₈ cycloalkyl, phenyl, C_{1-C}₆ alkoxy-(C₁-C₆)-alkyl, R³R³N—(C₁-C₆)-alkyl, fluoro-(C₁-C₆)-alkyl, —CONR³R³, —COOR³ or C₂-C₅ alkanoyl, and optionally substituted on a ring carbon atom not adjacent to a ring nitrogen atom by fluoro-(C₁-C₆)-alkoxy, halo, —OR³, cyano, —S(O)ₘR⁴, —NR³R³, —SO₂NR³R³, —NR³COR⁴ or —NR³SO₂R⁴, and said piperazin-1-yl and homopiperazin-1-yl being optionally substituted on the ring nitrogen atom not attached to A by C₁-C₆ alkyl, phenyl, C₁-C₆ alkoxy-(C₂-C₆)-alkyl, R³R³N—(C₂-C₆)-alkyl, fluoro-(C₁-C₆)-alkyl, C₂-C₅ alkanoyl, —COOR⁴, C₃-C₈ cycloalkyl, —SO₂R⁴, —SO₂NR³R³ or —CONR³R³,

or, R⁷ is H, C₁-C₆ alkyl, C₃-C₈ cycloalkyl, phenyl or benzyl and R⁸ is H, C₁-C₆ alkyl, C₃-C₈ cycloalkyl, phenyl, benzyl, fluoro-(C₁-C₆)-alkyl, —CONR³R³, —COOR⁴, C₂-C₅ alkanoyl or —SO₂NR³R³;

m is 0, 1 or 2; and

“het”, used in the definitions of R⁵ and R⁶, means C-linked pyrrolyl, imidazolyl, triazolyl, thienyl, furyl, thiazolyl, oxazolyl, thiadiazolyl, oxadiazolyl, pyridinyl, pyrimidinyl, pyridazinyl, pyrazinyl, quinolinyl, isoquinolinyl, benzimidazolyl, quinazolinyl, phthalazinyl, benzoxazolyl or quinoxalinyl, each optionally substituted by C₁-C₆ alkyl, C₁-C₆ alkoxy, cyano or halo.

WO-A-01/60835 discloses a compound of the formula:

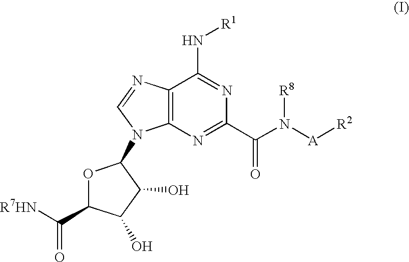

or a pharmaceutically acceptable salt or solvate thereof, wherein R¹ is hydrogen, C₁-C₆ alkyl or C₃-C₇ cycloalkyl, each optionally substituted by 1 or 2 substituents each independently selected from hydroxyl, fluorenyl, phenyl and naphthyl, said phenyl and naphthyl being optionally substituted by C₁-C₆ alkyl, C₁-C₆ alkoxy, halo or cyano;

A is a bond or C₁-C₆ alkylene;

R² is (i) hydrogen, C₁-C₆ alkyl, C₃-C₇ cycloalkyl, phenyl or naphthyl, said C₃-C₇ cycloalkyl, phenyl and naphthyl being optionally substituted by C₁-C₆ alkyl, phenyl, C₁-C₆ alkoxy-(C₁-C₆)-alkyl, amino-(C_{1-C}₆)-alkyl, fluoro-(C₁-C₆)-alkyl, fluoro-(C₁-C₆)-alkoxy, C₂-C₅ alkanoyl, halo, —OR³, cyano, —COOR³, C₃-C₇ cycloalkyl, —S(O)ₘR⁴ —NR³R³ —SO₂NR³R³ —CONR³R³—NR³COR⁴ or —NR³SO₂R⁴ with the proviso that R² is not hydrogen when A is a bond,

or (ii) when A is C₂-C₆ alkylene, —NR³R³, —OR³, —COOR³, —OCOR⁴, —SO₂R⁴₄ —CN, —SO₂NR³R³, —NR³SO₂R⁴, —NR³COR⁴ or —CONR³R³,

or (iii) a C-linked, 4 to 11 membered, mono or bicyclic heterocycle having either from 1 to 4 ring nitrogen atom(s) or 1 or 2 nitrogen and 1 oxygen or 1 sulphur ring atoms, optionally C-substituted by oxo, C₁-C₆ alkoxy-(C₁-C₆)-alkyl, amino-(C₁-C₆)-alkyl, fluoro-(C₁-C₆)-alkyl, fluoro-(C₁-C₆)-alkoxy, fluoro-(C₂-C₅)-alkanoyl, halo, cyano, —OR⁵, R⁶, —COR⁵, —NR⁵R⁵, —COOR⁵—S(O)ₘR⁶, —SO₂NR⁵R⁵, —CONR⁵R⁵, —NR⁵SO₂R⁶ or —NR⁵COR⁶ and optionally N-substituted by C₁-C₆ alkoxy-(C₁-C₆)-alkyl, amino-(C₂-C₆)-alkyl, fluoro-(C₁-C₆)-alkyl, fluoro-(C₂-C₅)-alkanoyl, R⁶, —COR⁵, —COOR⁶, —SO₂R⁶ —SO₂NR⁵R⁵ or —CONR⁵R⁵,

or (iv) when A is C₂-C₆ alkylene, N-linked azetidinyl, pyrrolidinyl, morpholinyl, tetrahydroisoquinolinyl, piperidinyl or piperazinyl, each being optionally C-substituted by C₁-C₆ alkyl, phenyl, C₁-C₆ alkoxy-(C₁-C₆)-alkyl, amino-(C₁-C₆)-alkyl, fluoro-(C₁-C₆)-alkyl, fluoro-(C₁-C₆)-alkoxy, C₂-C₅ alkanoyl, halo, —OR³, cyano, —COOR³, C₃-C₇ cycloalkyl, —S(O)ₘR⁴ —NR³R³, —SO₂NR³R³, —CONR³R³, —NR³COR⁴ or —NR³SO₂R⁴ and said piperazinyl being optionally N— substituted by C₁-C₆ alkyl, phenyl, C₁-C₆ alkoxy-(C₁-C₆)-alkyl, amino-(C₂-C₆)-alkyl, fluoro-(C₁-C₆)-alkyl, C₂-C₅ alkanoyl, —COOR⁴, C₃-C₇ cycloalkyl, —SO₂R⁴—SO₂NR³R³ or —CONR³R³;

each R³ is independently selected from H, C₁-C₆ alkyl, phenyl or pyridinyl;

R⁴ is C₁-C₆ alkyl or phenyl;

R⁵ is H, C₁-C₆ alkyl, C₃-C₇ cycloalkyl, phenyl, naphthyl or het;

R⁶ is C₁-C₆ alkyl, C₃-C₇ cycloalkyl, phenyl, naphthyl or het;

m is 0, 1 or 2;

R⁷ is hydrogen, C₁-C₆ alkyl, C₃-C₇ cycloalkyl, phenyl, naphthyl, azetidin-3-yl, pyrrolidin-3-yl, piperidin-3-yl, piperidin-4-yl or het, said azetidin-3-yl, pyrrolidin-3-yl, piperidin-3-yl and piperidin-4-yl being optionally substituted by C₁-C₆ alkyl;

R⁸ is H or C₁-C₆ alkyl; and

“het”, used in the definitions of R⁵, R⁶ and R⁷, means C-linked pyrrolyl, imidazolyl, triazolyl, thienyl, furyl, thiazolyl, oxazolyl, thiadiazolyl, oxadiazolyl, pyridinyl, pyrimidinyl, pyridazinyl, pyrazinyl, quinolinyl, isoquinolinyl, benzimidazolyl, quinazolinyl, phthalazinyl, benzoxazolyl or quinoxalinyl, each being optionally substituted by C₁-C₆ alkyl, C₁-C₆ alkoxy, cyano or halo.

WO-A-02/00676 discloses a compound of the formula

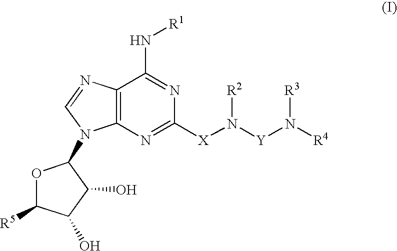

or a pharmaceutically acceptable salt or solvate thereof, wherein

R¹ is (i) H, (ii) C₁-C₆ alkyl optionally substituted by 1 or 2 substituents each independently selected from phenyl, naphthyl and fluorenyl, said phenyl, naphthyl and fluorenyl being optionally substituted by C₁-C₆ alkyl, C₁-C₆ alkoxy, halo or cyano, or (iii) fluorenyl;

R² is H or C₁-C₆ alkyl;

either, R³ and R⁴, taken together with the nitrogen atom to which they are attached, represent azetidinyl, pyrrolidinyl, piperidinyl, piperazinyl, homopiperidinyl or homopiperazinyl, each being optionally substituted on a ring nitrogen or carbon atom by C₁-C₆ alkyl or C₃-C₈ cycloalkyl and optionally substituted on a ring carbon atom not adjacent to a ring nitrogen atom by —NR⁶R⁷ or —OR⁹,

or, R³ is H, C₁-C₆ alkyl, C₃-C₈ cycloalkyl or benzyl, said C₁-C₆ alkyl being optionally substituted by C₃-C₈ cycloalkyl, and R⁴ is

(a) C₁-C₆ alkyl, C₃-C₈ cycloalkyl or R¹⁵, said C₁-C₆ alkyl being optionally substituted by R¹⁵, or

(b) —(C₂-C₆ alkylene)—R⁸, or

(c) —(C₁-C₆ alkylene)—R¹³;

R⁵ is —CH₂OH or —CONR¹⁴ R¹⁴;

R⁶ and R⁷ are either each independently H or C₁-C₆ alkyl or, taken together with the nitrogen atom to which they are attached, represent azetidinyl, pyrrolidinyl or piperidinyl, said azetidinyl, pyrrolidinyl and piperidinyl being optionally substituted by C₁-C₆ alkyl;

R⁸ is (i) azetidin-1-yl, pyrrolidin-1-yl, piperidin-1-yl, morpholin-4-yl, piperazin-1-yl, homopiperidin-1-yl, homopiperazin-1-yl or tetrahydroisoquinolin-1-yl, each being optionally substituted on a ring carbon atom by C₁-C₆ alkyl, C₃-C₈ cycloalkyl, phenyl, C₁-C₆ alkoxy-(C₁-C₆)-alkyl, R⁹R⁹N—(C₁-C₆)-alkyl, fluoro-(C₁-C₆)-alkyl, —CONR⁹R⁹, —COOR⁹ or C₂-C₅ alkanoyl and optionally substituted on a ring carbon atom not adjacent to a ring nitrogen atom by fluoro-(C₁-C₆)-alkoxy, halo, —OR⁹, cyano, —S(O)ₘR¹⁰, —NR⁹R⁹, —SO₂NR⁹R⁹, —NR⁹COR¹⁰ or —NR⁹SO₂R¹⁰ and said piperazin-1-yl and homopiperazin-1-yl being optionally substituted on the ring nitrogen atom not attached to the C₂-C₆ alkylene group by C₁-C₆ alkyl, phenyl, C₁-C₆ alkoxy-(C₂-C₆)-alkyl, R⁹R⁹N—(C₂-C₆)-alkyl, fluoro-(C₁-C₆)-alkyl, C₂-C₅ alkanoyl, —COOR¹⁰, C₃-C₈ cycloalkyl, —SO₂R¹⁰, —SO₂NR⁹R⁹ or —CONR⁹R⁹, or

(ii) —NR¹¹ R¹²;

R⁹ is H, C₁-C₆ alkyl, C₃-C₈ cycloalkyl or phenyl;

R¹⁰ is C₁-C₆ alkyl, C₃-C₈ cycloalkyl or phenyl;

R¹¹ is C₁-C₆ alkyl, C₃-C₈ cycloalkyl or benzyl;

R¹² is C₁-C₆ alkyl, C₃-C₈ cycloalkyl, phenyl, benzyl, fluoro-(C₁-C₆)-alkyl, —CONR⁹R⁹, —COOR¹⁰, —COR¹⁰, —SO₂R¹⁰ or —SO₂NR⁹R⁹, said C₁-C₆ alkyl being optionally substituted by phenyl;

R¹³ is phenyl, pyridin-2-yl, pyridin-3-yl or pyridin-4-yl, each being optionally substituted by C₁-C₆ alkyl, C₁-C₆ alkoxy, halo or cyano;

R¹⁴ is H or C₁-C₆ alkyl optionally substituted by cyclopropyl;

R¹⁵ is azetidin-3-yl, pyrrolidin-3-yl, piperidin-3-yl, piperidin-4-yl, homopiperidin-3-yl or homopiperidin-4-yl, each being optionally substituted by R¹³, C₁-C₆ alkyl, C₃-C₈ cycloalkyl or benzyl;

m is 0, 1 or2;

X is —CH₂— or —CH₂CH₂—; and

Y is CO, CS, SO₂ or C═N(CN).

WO-A-01/94368 discloses a compound of the formula

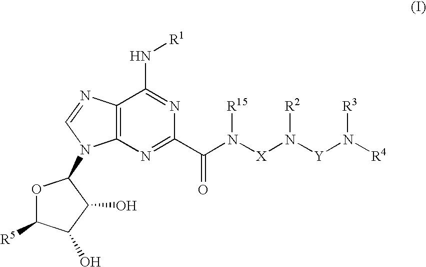

or a pharmaceutically acceptable salt or solvate thereof, wherein

R¹ is H, C₁-C₆ alkyl or fluorenyl, said C₁-C₆ alkyl being optionally substituted by 1 or 2 substituents each independently selected from phenyl and naphthyl, said phenyl and naphthyl being optionally substituted by C₁-C₆ alkyl, C₁-C⁶ alkoxy, halo or cyano;

(A) R² is H or C₁-C₆ alkyl, R¹⁵ is H or C₁-C₆ alkyl, and X is either (i) unbranched C₂-C₃ alkylene optionally substituted by C₁-C₆ alkyl or C₃-C₈ cycloalkyl, or (ii) a group of the formula:

—(CH₂)ₙ—W—(CH₂)ₚ—

where W is C₅-C₇ cycloalkylene optionally substituted by C₁-C₆ alkyl, n is 0 or 1 and p is 0 or 1, or

(B) R¹⁵ is H or C₁-C₆ alkyl, and R² and X, taken together with the nitrogen atom to which they are attached, represent azetidin-3-yl, pyrrolidin-3-yl, piperidin-3-yl, piperidin-4-yl, homopiperidin-3-yl or homopiperidin-4-yl, each being optionally substituted by C₁-C₆ alkyl, or

(C) R² is H or C₁-C₆ alkyl, and R¹⁵ and X, taken together with the nitrogen atom to which they are attached, represent azetidin-3-yl, pyrrolidin-3-yl, piperidin-3-yl, piperidin-4-yl, homopiperidin-3-yl or homopiperidin-4-yl, each being optionally substituted by C₁-C₆ alkyl;

either, R³ and R⁴, taken together with the nitrogen atom to which they are attached, represent azetidinyl, pyrrolidinyl, piperidinyl, piperazinyl, homopiperidinyl or homopiperazinyl, each being optionally substituted on a ring nitrogen or carbon atom by C₁-C₆ alkyl or C₃-C₈ cycloalkyl and optionally substituted on a ring carbon atom not adjacent to a ring nitrogen atom by —NR⁶R⁷,

or, R³ is H, C₁-C₆ alkyl, C₃-C₈ cycloalkyl or benzyl and R⁴ is

(a) azetidin-3-yl, pyrrolidin-3-yl, piperidin-3-yl, piperidin-4-yl, homopiperidin-3-yl or homopiperidin-4-yl, each being optionally substituted by C₁-C₆ alkyl, C₃-C₈ cycloalkyl, phenyl, benzyl or het, or

(b) —(C₂-C₆ alkylene)—R⁸,

(c) —(C₁-C₆ alkylene)—R¹³, or

(d) C₁-C₆ alkyl or C₃-C₈ cycloalkyl;

R⁵ is CH₂OH or CONR¹⁴R¹⁴;

R⁶ and R⁷ are either each independently H or C₁-C₆ alkyl or, taken together with the nitrogen atom to which they are attached, represent azetidinyl, pyrrolidinyl or piperidinyl, said azetidinyl, pyrrolidinyl and piperidinyl being optionally substituted by C₁-C₆ alkyl;

R⁸ is (i) azetidin-1-yl, pyrrolidin-1-yl, piperidin-1-yl, morpholin-4-yl, piperazin-1-yl, homopiperidin-1-yl, homopiperazin-1-yl or tetrahydroisoquinolin-1-yl, each being optionally substituted on a ring carbon atom by C₁-C₆ alkyl, C₃-C₈ cycloalkyl, phenyl, C₁-C₆ alkoxy-(C₁-C₆)-alkyl, R⁹R⁹N—(C₁-C₆)-alkyl, fluoro-(C₁-C₆)-alkyl, —CONR⁹R⁹, —COOR⁹ or C₂-C₅ alkanoyl, and optionally substituted on a ring carbon atom not adjacent to a ring nitrogen atom by fluoro-(C₁-C₆)-alkoxy, halo, —OR⁹, cyano, —S(O)ₘR¹⁰, —NR⁹R⁹, —SO₂NR⁹R⁹, —NR⁹COR¹⁰ or —NR⁹SO₂R¹⁰, and said piperazin-1-yl and homopiperazin-1-yl being optionally substituted on the ring nitrogen atom not attached to the C₂-C₆ alkylene group by C₁-C₆ alkyl, phenyl, C₁-C₆ alkoxy-(C₂C₆)-alkyl, R⁹R⁹N—(C₂-C₆)-alkyl, fluoro-(C₁-C₆)-alkyl, C₂-C₅ alkanoyl, —COOR¹⁰, C₃-C₈ cycloalkyl, —SO₂R₁₀, —SO₂NR⁹R⁹ or —CONR⁹R⁹, or

(ii) NR¹¹R¹²;

R⁹ is H, C₁-C₆ alkyl, C₃-C₈ cycloalkyl or phenyl;

R¹⁰ is C₁-C₆ alkyl, C₃-C₈ cycloalkyl or phenyl;

R¹¹ is H, C₁-C₆ alkyl, C₃-C₈ cycloalkyl or benzyl;

R¹² is H, C₁-C₆ alkyl, C₃-C₈ cycloalkyl, phenyl, benzyl, fluoro-(C₁-C₆)-alkyl, —CONR⁹R⁹, —COOR¹⁰, C₂-C₅ alkanoyl or —SO₂NR⁹R⁹;

R¹³ is (a) phenyl, pyridin-2-yl, pyridin-3-yl or pyridin-4-yl, each being optionally substituted by C₁-C₆ alkyl, C₁-C₆ alkoxy, —(C₁-C₃ alkylene)-(C₁-C₆ alkoxy), halo, cyano, —(C₁-C₃ alkylene)—CN, —CO₂H, —(C₁-C₃ alkylene)—CO₂H, —CO₂(C₁-C₆ alkyl), —(C₁-C₃ alkylene)—CO₂(C₁-C₆ alkyl), —(C₁-C₃ alkylene)—NR¹⁴R¹⁴—CONR¹⁴R¹⁴ or —(C₁-C₃ alkylene)—CONR¹⁴R¹⁴, or (b) azetidin-2-yl, azetidin-3-yl, pyrrolidin-2-yl, pyrrolidin-3-yl, piperidin-2-yl, piperidin-3-yl, piperidin-4-yl, homopiperidin-2-yl, homopiperidin-3-yl or homopiperidin-4-yl, each being optionally substituted by C₁-C₆ alkyl, C₃-C₈ cycloalkyl, phenyl, benzyl or het;

R¹⁴ is H or C₁-C₆ alkyl optionally substituted by cyclopropyl;

m is 0, 1 or 2;

Y is CO, CS, SO₂ or C═N(CN); and

“het”, used in the definition of R⁴ and R¹³, is a C-linked, 4- to 6-membered ring, heterocycle having either from 1 to 4 ring nitrogen heteroatoms or 1 or 2 nitrogen ring heteroatoms and 1 oxygen or 1 sulphur ring heteroatom, optionally substituted by C₁-C₆ alkyl, C₃-C₈ cycloalkyl, C₁-C₆ alkoxy, C₃-C₈ cycloalkoxy, hydroxy, oxo or halo.

Preferred selective adenosine A₂ₐ-receptor agonists for use in the invention include:

N-({9-[(2R,3R,4S,5R)-3,4-dihydroxy-5-(methoxymethyl)tetrahydro-2-furanyl]-6-[(2,2-diphenylethyl)amino]-9H-purin-2-yl}methyl-2-methyl-1-propanesulfonamide (Example 15 of WO-A-00/23457);

cis —(2R,3R,4S,5R)-2-(6-[(2,2-diphenylethyl)amino]-2-{[(4-isopropylcyclohexyl)amino]methyl}-9H-purin-9-yl)-5-(methoxymethyl)tetrahydro-3,4-furandiol and trans-(2R,3R,4S,5R)-2-(6-[(2,2-diphenylethyl)amino]-2-{[(4-isopropylcyclohexyl)amino]methyl}-9H-purin-9-yl)-5-(methoxymethyl)tetrahydro-3,4-furandiol (Example 17 of WO-A-00/23457);

N-({9-[(2R,3R,4S,5R)-3,4-dihydroxy-5-(hydroxymethyl)tetrahydro-2-furanyl]-6-[(2,2-diphenylethyl)amino]-9H-purin-2-yl}methyl)-2-methyl-1-propanesulfonamide (Example 1 of WO-A-01/27130);

(2S,3S,4R,5R)-5-(6-[(2,2-diphenylethyl)amino]-2-{[(isopropylsulfonyl)amino]methyl}-9H-purin-9-yl)-N-ethyl-3,4-dihydroxytetrahydro-2-furancarboxamide (Example 3 of WO-A-01/27131);

9-[(2R,3R,4S,5R)-3,4-dihydroxy-5-(hydroxymethyl)tetrahydro-2-furanyl]-6-[(2,2-diphenylethyl)amino]-N-[2-(1-piperidinyl)ethyl]-9H-purine-2-carboxamide (Example 1 of WO-A-00/77018);

6-[(2,2-diphenylethyl)amino]-9-{(2R,3R,4S,5S)-5-[(ethylamino)carbonyl]-3,4-dihydroxytetrahydro-2-furanyl}-N-[2-(1-piperidinyl)ethyl]-9H-purine-2-carboxamide (Example 1 of WO-A-01/60835);

N-({9-[(2R,3R,4S,5R)-3,4-dihydroxy-5-(hydroxymethyl)tetrahydro-2-furanyl]-6-[(2,2-diphenylethyl)amino]-9H-purin-2-yl}methyl)-N′-[2-(diisopropylamino)ethyl]urea (Example 1 of WO-A-02/00676); and

6-[(2,2-diphenylethyl)amino]-9-{(2R,3R,4S,5S)-5-[(ethylamino)carbonyl]-3,4-dihydroxytetrahydro-2-furanyl}-N{2-[({[1-(2-pyridinyl)-4-piperidinyl]amino}carbonyl)amino]ethyl}-9H-purine-2-carboxamide (Examples 8 and 35 of WO-A-01/94368); and the pharmaceutically acceptable salts and solvates thereof.

Particularly preferred selective adenosine A₂ₐ-receptor agonists for use in the invention include 9-[(2R,3R,4S,5R)-3,4-dihydroxy-5-(hydroxymethyl)tetrahydro-2-furanyl]-6-[(2,2-diphenylethyl)amino]-N-[2-(1-piperidinyl)ethyl]-9H-purine-2-carboxamide and 6-[(2,2-diphenylethyl)amino]-9-{(2R,3R,4S,5S)-5-[(ethylamino)carbonyl]-3,4-dihydroxytetrahydro-2-furanyl}-N-{2-[({[1-(2-pyridinyl)-4-piperidinyl]amino}carbonyl)amino]ethyl}-9H-purine-2-carboxamide and the pharmaceutically acceptable salts and solvates thereof. Most preferred is 6-[(2,2-diphenylethyl)amino]-9-{(2R,3R,4S,5S)-5-[(ethylamino)carbonyl]-3,4-dihydroxytetrahydro-2-furanyl}-N-(2-[({[1-(2-pyridinyl)-4-piperidinyl]amino}carbonyl)amino]ethyl}-9H-purine-2-carboxamide and the pharmaceutically acceptable salts and solvates thereof.

Suitable anticholinergic agents for use in the invention include ipratropium and oxitropium salts and solvates thereof.

A tiotropium salt (see EP418716 B1) has the structure of formula (1.1):

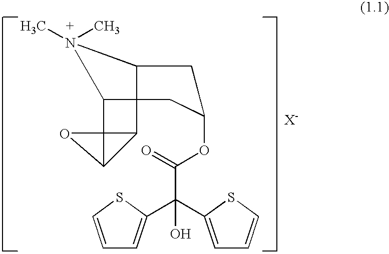

wherein X^{—} is a physiologically acceptable anion.

An ipratropium salt (see EP309464 B1) has the structure of formula (1.2):

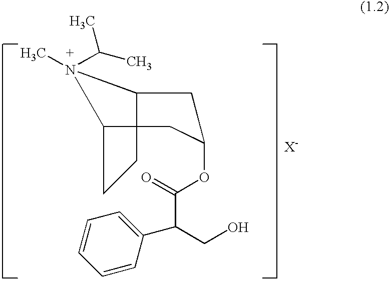

wherein X^{—} is a physiologically acceptable anion.

An oxitropium salt (see EP579615 B1) has the structure of formula (1.3):

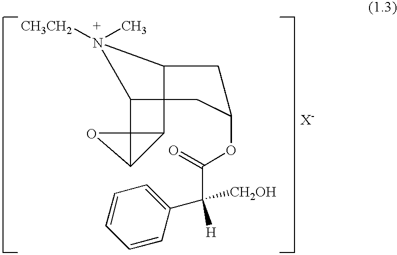

wherein X^{—} is a physiologically acceptable anion.

Examples of suitable salt forms of ipratropium and oxitropium are fluoride, F^{—}; chloride, Cl^{—}; bromide, Br^{—}; iodide, I^{—}; methanesulfonate, CH₃S(═O)₂O^{—}; ethanesulfonate, CH₃CH₂S(═O)₂O^{—}; methylsulfate, CH₃OS(═O)₂O^{—}; benzene sulfonate, C₆H₅S(═O)₂O^{—}; and p-toluenesulfonate, 4-CH₃-C₆H₅S(═O)₂O^{—}. The bromide salt form is preferred.

Preferred specific combinations of a selective adenosine A₂ₐ receptor agonist and an anticholinergic compound according to the invention include:

9-[(2R,3R,4S,5R)-3,4-dihydroxy-5-(hydroxymethyl)tetrahydro-2-furanyl]-6-[(2,2-diphenylethyl)amino]-N-[2-(1-piperidinyl)ethyl]-9H-purine-2-carboxamide or a pharmaceutically acceptable salt or solvate thereof and an ipratropium salt, or solvate thereof;

6-[(2,2-diphenylethyl)amino]-9{-(2R,3R,4S,5S)-5-[(ethylamino)carbonyl]-3,4-dihydroxytetrahydro-2-furanyl}-N-{2-[({[1-(2-pyridinyl)-4-piperidinyl]amino}carbonyl)amino]ethyl}-9H-purine-2-carboxamide or a pharmaceutically acceptable salt or solvate thereof and an ipratropium salt, or solvate thereof;

9-[(2R,3R,4S,5R)-3,4-dihydroxy-5-(hydroxymethyl)tetrahydro-2-furanyl]-6-[(2,2-diphenylethyl)amino]-N-[2-(1-piperidinyl)ethyl]-9H-purine-2-carboxamide or a pharmaceutically acceptable salt or solvate thereof and an oxitropium salt, or solvate thereof; and

6-[(2,2-diphenylethyl)amino]-9-{(2R,3R,4S,5S)-5-[(ethylamino)carbonyl]-3,4-dihydroxytetrahydro-2-furanyl}-N-{2-[({[1-(2-pyridinyl)-4-piperidinyl]amino}carbonyl)amino]ethyl}-9H-pudne-2-carboxamide or a pharmaceutically acceptable salt or solvate thereof and an oxitropium salt, or solvate thereof.

A selective adenosine A₂ₐ receptor agonist or an anticholinergic agent used in accordance with the invention may optionally be utilised in the form of a pharmaceutically acceptable salt or solvate. Such a salt may be an acid addition or a base salt.

Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts.

Suitable base salts are formed from bases which form non-toxic salts and examples are the sodium, potassium, aluminium, calcium, magnesium, zinc and diethanolamine salts.

For a review on suitable salts see Berge et al, J. Pharm. Sci., 66, 1-19, 1977.

The pharmaceutically acceptable solvates of the selective adenosine A₂ₐ receptor agonists and anticholinergic agents used in accordance with the invention, or salts thereof, include the hydrates thereof.

The selective adenosine A₂ₐ receptor agonists and anticholinergic agents of the invention may exist in one or more polymorphic forms.

The selective adenosine A₂ₐ receptor agonists and anticholinergic agents of the invention may contain one or more asymmetric carbon atoms and therefore exists in two or more stereoisomeric forms. Where such a compound contains an alkenyl or alkenylene group, cis/trans (or Z/E) isomerism may also occur. The present invention includes these individual stereoisomers of the compounds of the invention and, where appropriate, the individual tautomeric forms thereof, together with mixtures thereof.

Separation of diastereoisomers or cis and trans isomers may be achieved by conventional techniques, e.g. by fractional crystallisation, chromatography or H.P.L.C. of a stereoisomeric mixture of a compound of the invention or a suitable salt or derivative thereof. An individual enantiomer of a compound of the invention may also be prepared from a corresponding optically pure intermediate or by resolution, such as by H.P.L.C. of the corresponding racemate using a suitable chiral support or by fractional crystallisation of the diastereoisomeric salts formed by reaction of the corresponding racemate with a suitable optically active acid or base, as appropriate.

The present invention also includes all suitable isotopic variations of a compound of the invention or a pharmaceutically acceptable salt thereof. An isotopic variation of a compound of the invention or a pharmaceutically acceptable salt thereof is defined as one in which at least one atom is replaced by an atom having the same atomic number but an atomic mass different from the atomic mass usually found in nature. Examples of isotopes that can be incorporated into compounds of the invention and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the compounds of the invention and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances.

The types of diseases that may be treated using the combinations of the present invention include, but are not limited to, asthma, chronic or acute bronchoconstriction, chronic bronchitis, small airways obstruction, emphysema, chronic obstructive pulmonary disease (COPD), COPD that has chronic bronchitis, pulmonary emphysema or dyspnea associated therewith and COPD that is characterised by irreversible, progressive airways obstruction.

Asthma

One of the most important respiratory diseases treatable with the combinations of therapeutic agents of the present invention is asthma, a chronic, increasingly common disorder encountered worldwide and characterized by intermittent reversible airway obstruction, airway hyper-responsiveness and inflammation. The cause of asthma has yet to be determined, but the most common pathological expression of asthma is inflammation of the airways, which may be significant even in the airways of patients with mild asthma. This inflammation drives reflex airway events resulting in plasma protein extravasation, dyspnea and bronchoconstriction. Based on bronchial biopsy and lavage studies it has been clearly shown that asthma involves infiltration by mast cells, eosinophils, and T-lymphocytes into a patient's airways. Bronchoalveolar lavage (BAL) in atopic asthmatics shows activation of interleukin (IL)-3, IL-4, IL-5 and granulocyte/macrophage-colony stimulating factor (GM-CSF) that suggests the presence of a T-helper 2 (Th-2)-like T-cell population.

The combinations of therapeutic agents of the present invention are useful in the treatment of atopic and non-atopic asthma. The term “atopy” refers to a genetic predisposition toward the development of type I (immediate) hypersensitivity reactions against common environmental antigens. The most common clinical manifestation is allergic rhinitis, while bronchial asthma, atopic dermatitis, and food allergy occur less frequently. Accordingly, the expression “atopic asthma” as used herein is intended to be synonymous with “allergic asthma”, i.e., bronchial asthma which is an allergic manifestation in a sensitized person. The term “non-atopic asthma” as used herein is intended to refer to all other asthmas, especially essential or “true” asthma, which is provoked by a variety of factors, including vigorous exercise, irritant particles, psychologic stresses, etc.

Chronic Obstructive Pulmonary Disease (COPD)

The combinations of therapeutic agents of the present invention are useful in the treatment of COPD or COAD including chronic bronchitis, pulmonary emphysema or dyspnea associated therewith. COPD is characterized by poorly reversible, progressive airways obstruction. Chronic bronchitis is associated with hyperplasia and hypertrophy of the mucus secreting glands of the submucosa in the large cartilaginous airways. Goblet cell hyperplasia, mucosal and submucosal inflammatory cell infiltration, edema, fibrosis, mucus plugs and increased smooth muscle are all found in the terminal and respiratory bronchioles. The small airways are known to be a major site of airway obstruction. Emphysema is characterized by destruction of the alveolar wall and loss of lung elasticity. A number of risk factors have also been identified as linked to the incidence of COPD. The link between tobacco smoking and COPD is well established. Other risk factors include exposure to coal dust and various genetic factors. See Sandford et al., “Genetic risk factors for chronic obstructive pulmonary disease,” Eur. Respir. J. 10 1380-1391, 1997. The incidence of COPD is increasing and it represents a significant economic burden on the populations of the industrialized nations. COPD also presents itself clinically with a wide range of variation from simple chronic bronchitis without disability to patients in a severely disabled state with chronic respiratory failure.

COPD is characterized by inflammation of the airways, as is the case with asthma, but the inflammatory cells that have been found in the bronchoalveolar lavage fluid and sputum of patients are neutrophils and macrophages rather than eosinophils. Elevated levels of inflammatory mediators are also found in COPD patients, including IL-8, LTB₄, and TNF-_{α}, and the surface epithelium and sub-epithelium of the bronchi of such patients has been found to be infiltrated by T-lymphocytes and macrophages. Symptomatic relief for COPD patients can be provided by the use of p-agonist and anticholinergic bronchodilators, but the progress of the disease remains unaltered. COPD has been treated using theophylline, but without much success, due in part to its propensity to produce unwanted effects. Steroids have also failed to hold out much promise as satisfactory treatment agents in COPD as they are relatively ineffective as anti-inflammatory agents.

Accordingly, the use of the combinations of therapeutic agents of the present invention to treat COPD and its related and included obstructed airways diseases, represents a significant advance in the art. The present invention is not limited to any particular mode of action or any hypothesis as to the way in which the desired therapeutic objectives have been obtained by utilizing the combinations of therapeutic agents of the present invention.

Bronchitis and Bronchiectasis

In accordance with the particular and diverse inhibitory activities described above that are possessed by the combinations of therapeutic agents of the present invention, they are useful in the treatment of bronchitis of whatever type, etiology, or pathogenesis, including, e.g., acute bronchitis which has a short but severe course and is caused by exposure to cold, breathing of irritant substances, or an acute infection; catarrhal bronchitis which is a form of acute bronchitis with a profuse mucopurulent discharge; chronic bronchitis which is a long-continued form of bronchitis with a more or less marked tendency to recurrence after stages of quiescence, due to repeated attacks of acute bronchitis or chronic general diseases, characterized by attacks of coughing, by expectoration either scanty or profuse, and by secondary changes in the lung tissue; dry bronchitis which is characterized by a scanty secretion of tough sputum; infectious asthmatic bronchitis which is a syndrome marked by the development of symptoms of bronchospasm following respiratory tract infections in persons with asthma; productive bronchitis which is bronchitis associated with a productive cough.

The use of the combinations of therapeutic agents of the present invention to treat atopic asthma or non-atopic asthma, COPD or other chronic inflammatory airways diseases may be established and demonstrated by use of a number of different models known in the art of inhibition of reflex events in the airway including plasma extravasation and bronchospasmolytic models described below.

Bronchodilator Activity—cAMP is involved not only in smooth muscle relaxation, but also exerts an overall inhibitory influence on airway smooth muscle proliferation, both of which may result from activation of A2a receptors by a component of the invention. Airway smooth muscle hypertrophy and hyperplasia can be modulated by cAMP, and these conditions are common morphological features of chronic asthma.

Bronchospasmolytic Activity In Vitro—The ability of the combinations of therapeutic agents of the present invention to cause relaxation of guinea-pig tracheal smooth muscle is demonstrated in the following test procedure. Guinea-pigs (350-500 g) are killed with sodium pentothal (100 mg/kg i.p.). The trachea is dissected and a section 2-3 cm in length is excised. The trachea is transected in the transverse plane at alternate cartilage plates so as to give rings of tissue 3-5 mm in depth. The proximal and distal rings are discarded. Individual rings are mounted vertically on stainless steel supports, one of which is fixed at the base of an organ bath, while the other is attached to an isometric transducer. The rings are bathed in Krebs solution (composition μM: NaHCO₃ 25; NaCl 113; KCl 4.7; MgSO₄. 7H₂O 1.2; KH₂PO₄ 1.2; CaCl₂ 2.5; glucose 11.7) at 37° C. and gassed with O₂/CO₂ (95:5, v/v). Rings prepared in this manner are contracted by field stimulation. To ascertain spasmolytic activity, test combinations of therapeutic agents of the present invention are dissolved in physiological saline and added in increasing quantities to the organ bath at 5 m intervals to provide a cumulative concentration-effect curve.

In the above test model, combinations of therapeutic agents of the present invention inhibit field stimulated contraction of guinea-pig tracheal ring preparations at concentrations in the range of from 0.001 to 1.0 μM.

Relaxation of Human Bronchus—Samples of human lungs dissected during surgery for cancer are obtained within 3 days after removal. Small bronchi (inner diameter ≈2 to 5 mm) are excised, cut into segments and placed in 2 ml liquid nitrogen storage ampoules filled with fetal calf serum (FCS) containing 1.8M dimethylsulfoxide (DMSO) and 0.1M sucrose as cryoprotecting agents. The ampoules are placed in a polystyrol box (11×11×22 cm) and slowly frozen at a mean cooling rate of about 0.6° C./m in a freezer maintained at −70° C. After 3-15h the ampoules are transferred into liquid nitrogen (−196° C.) where they are stored until use. Before use the tissues are exposed for 30-60 m to −70° C. before being thawed within 2.5 m by placing the ampoules in a 37° C. water bath. Thereafter the bronchial segments are rinsed by placing them in a dish containing Krebs-Henseleit solution (μM: NaCl 118, KCl 4.7. MgSO₄ 1.2, CaCl₂ 1.2, KH₂PO₄ 1.2, NaHCO₃ 25, glucose 11, EDTA 0.03) at 37° C., cut into rings and suspended in 10 ml organ baths for isometric tension recording under a preload of about 1 g. Further increases in tension are induced via the application of field stimulation, which is known to induce activation of nerves in the airway sample and generate tension via release of acetylcholine and other neurally derived mediators. Concentration-response curves are produced by cumulative additions, each concentration being added when the maximum effect has been produced by the previous concentration. Papaverine (300 μM) is added at the end of the concentration response curve to induce complete relaxation of the bronchial rings. This effect is taken as 100% relaxation.

In the above test model the combinations of therapeutic agents of the present invention produce concentration-related relaxation of human bronchus ring preparations at concentrations in the range of from 0.001 to 1.0 μM with preferred embodiments being active at concentrations in the range of from 5.0 nM to 500 nM.

Suppression of Capsaicin-induced Bronchoconstriction—Male Dunkin-Hartley guinea- pigs (400-800 g) having free access to food and water prior to the experiment, are anaesthetized with sodium phenobarbital (100 mg/kg i.p. [intra peritoneal]). Animals, maintained at 37° C. with a heated pad, controlled by a rectal thermometer, are ventilated via a tracheal cannula (about 8 ml/kg, 1 Hz) with a mixture of air and oxygen (45:55 v/v). Ventilation is monitored at the trachea by a pneumotachograph connected to a differential pressure transducer in line with the respiratory pump. Pressure changes within the thorax are monitored directly via an intrathoracic cannula, using a differential pressure transducer so that the pressure difference between the trachea and thorax can be measured and displayed. From these measurements of air-flow and transpulmonary pressure, both airway resistance (R₁ cmH₂O/l/s) and compliance (Cd_{dyn}) are calculated with a digital electronic respiratory analyzer for each respiratory cycle. Blood pressure and heart rate are recorded from the carotid artery using a pressure transducer.

When values for basal resistance and compliance are stable, an acute episode of bronchoconstriction is induced by an intravenous bolus of capsaicin. Capsaicin is dissolved in 100% ethanol and diluted with phosphate buffered saline. Test combinations of therapeutic agents of the present invention are administered when the response to capsaicin is stable, which is calculated to be after 2-3 such administrations at 10 min intervals. Reversal of bronchoconstriction is assessed over 1-8 h following either intratracheal or intraduodenal instillation or intravenous bolus injection. Bronchospasmolytic activity is expressed as a % inhibition of the initial, maximal resistance (RD) following the infusion of capsaicin. ED₅₀ values represent the dose which causes a 50% reduction of the increase in resistance induced by capsaicin. Duration of action is defined as the time in minutes where bronchoconstriction is reduced by 50% or more. Effects on blood pressure (BP) and heart rate (HR) are characterized by ED₂₀ values; i.e., the doses which reduce BP or HR by 20% measured 5 m after administration.

In the above test model the combinations of therapeutic agents of the present invention exhibit bronchodilator activity at dosages in the range of from 0.001 to 0.1 mg/kg i.t. [intra tracheal]. Further, the combination delivered i.t. exhibits an at least additive inhibitory effect on bronchospasm, with each component alone being able to inhibit more than 50% of the observed control response.

LPS-Induced Lung Neutrophilia—The recruitment to and activation of neutrophils in the lungs is considered an important pathological feature in COPD and in severe asthma. Consequently, inhibition of either or both of these endpoints in animals provides supportive evidence of the utility of the present invention.

Male Wistar-Albino rats (150-250 g) or male Dunkin-Hartley guinea-pigs (400-600 g) are pretreated with the test articles alone or in combination by inhalation or intratracheal (i.t.) instillation under brief general anaesthesia. After 1-24 h after compound administration, animals are challenged with an inhalation aerosol of bacterial liopolysaccharide (LPS) sufficient to induce over the subsequent 1-24 h of a pronounced lung neutrophilia. The neutrophilia is assessed by cell counting in bronchial washings or by determination of neutrophil products in lung washings or tissue. In this test system, the therapeutic agents of the present invention exhibit anti-inflammatory activity at doses ranging from 0.0001 to 0.1 mg/kg i.t. Unexpectedly, the combination delivered i.t. exerts at least an additive effect on inflammation, despite the fact that one of the components does not on its own exert a significant anti-inflammatory effect. Further, equivalent anti-inflammatory effects of a high dose of one of the components can be observed with lower doses when used in combination as in this invention, thus minimising systemic unwanted effects.

Allergic guinea-pig Assay—A test for evaluating the therapeutic impact of the combinations of therapeutic agents of the present invention on the symptom of dyspnea and bronchspasm i.e., difficult or labored breathing and increased lung resistance, and on the symptom of inflammation, ie: lung neutrophilia and eosinophilia, utilizes Dunkin-Hartley guinea-pigs (400-600 g body weight).

The egg albumin (EA), grade V, crystallized and lyophilized, aluminum hydroxide, and mepyramine maleate used in this test are commercially available. The challenge and subsequent respiratory readings are carried out in a clear plastic box with internal dimensions of 10×6×4 inches. The head and body sections of the box are separable. In use the two are held firmly together by clamps, and an airtight seal between the chambers is maintained by a soft rubber gasket. Through the centre of the head end of the chamber a nebulizer is inserted via an airtight seal and each end of the box also has an outlet. A pneumotachograph is inserted into one end of the box and is coupled to a volumetric pressure transducer which is then connected to a dynograph through appropriate couplers. While aerosolizing the antigen, the outlets are open and the pneumotachograph is isolated from the chamber. The outlets are then closed and the pneumotachograph and the chamber are connected during the recording of the respiratory patterns. For challenge, 2 ml of a 3% solution of antigen in saline is placed in each nebulizer and the aerosol is generated with air from a small diaphragm pump operating at 10 psi and a flow rate of 8 l/m.

Guinea-pigs are sensitized by injecting subcutaneously and i.p. 1 ml of a suspension containing 1 mg EA and 200 mg aluminum hydroxide in saline. They are used between days 12 and 24 post-sensitization. In order to eliminate the histamine component of the response, guinea-pigs are pretreated i.p. 30 min prior to aerosol challenge with 2 mg/kg of mepyarmine. Guinea-pigs are then exposed to an aerosol of 3% EA in saline for exactly 1 m, then respiratory profiles are recorded for a further 30 m. Subsequently, lung inflammation is determined post mortem over a period of 1-48 h. The duration of continuous dyspnea is measured from the respiratory recordings.

Test combinations of therapeutic agents of the present invention are generally administered i.t. or by aerosol 0.5-4 h prior to challenge. The combinations of compounds are either dissolved in saline or biocompatible solvents. The activity of the compounds is determined on the basis of their ability to decrease the magnitude and duration of symptoms of dyspnea and broncospasm and/or magnitude of lung inflammation in comparison to a group of vehicle-treated controls. Tests of the combinations of therapeutic agents of the present invention are evaluated over a series of doses and an ED₅₀ is derived that is defined as the dose (mg/kg) which will inhibit the duration of symptoms by 50%.

Anti-inflammatory Activity—The anti-inflammatory activity of the combinations of therapeutic agents of the present invention is demonstrated by the inhibition of eosinophil or neutrophil activation. In this assay blood samples (50 ml) are collected from non-atopic volunteers with eosinophil numbers ranging between 0.06 and 0.47×10⁹ L⁻¹. Venous blood is collected into centrifuge tubes containing 5 ml trisodium citrate (3.8%, pH 7.4).

The anticoagulated blood is diluted (1:1, v:v) with phosphate-buffered saline (PBS, containing neither calcium nor magnesium) and is layered onto 15 ml isotonic Percoll (density 1.082-1.085 g/ml, pH 7.4), in a 50 ml centrifuge tube. Following centrifugation (30 minutes, 1000×g, 20° C.), mononuclear cells at the plasma/Percoll interface are aspirated carefully and discarded.

The neutrophil/eosinophil/erythrocyte pellet (ca. 5 ml by volume) is gently resuspended in 35 ml of isotonic ammonium chloride solution (NH₄Cl, 155 mM; KHCO₃, 10 mM; EDTA. 0.1 mM; 0-4° C.). After 15 min, cells are washed twice (10 min, 400×g, 4° C.) in PBS containing fetal calf serum (2%, FCS).

A magnetic cell separation system is used to separate eosinophils and neutrophils. This system is able to separate cells in suspension according to surface markers, and comprises a permanent magnet, into which is placed a column that includes a magnetizable steel matrix. Prior to use, the column is equilibrated with PBS/FCS for 1 hour and then flushed with ice-cold PBS/FCS on a retrograde basis via a 20 ml syringe. A 21G hypodermic needle is attached to the base of the column and 1-2 ml of ice cold buffer are allowed to efflux through the needle. Following centrifugation of granulocytes, supernatant is aspirated and cells are gently resuspended with 100 μl magnetic particles (anti-CD16 monoclonal antibody, conjugated to superparamagnetic particles). The eosinophil/neutrophil/anti-CD16 magnetic particle mixture is incubated on ice for 40 minutes and then diluted to 5 ml with ice-cold PBS/FCS. The cell suspension is slowly introduced into the top of the column and the tap is opened to allow the cells to move slowly into the steel matrix. The column is then washed with PBS/FCS (35 ml), which is carefully added to the top of the column so as not to disturb the magnetically labeled neutrophils already trapped in the steel matrix. Non-labeled eosinophils are collected in a 50 ml centrifuge tube and washed (10 minutes, 400×g, 40° C.). The resulting pellet is resuspended in 5 ml Hank's balanced salt solution (HBSS) so that cell numbers and purity can be assessed prior to use. The separation column is removed from the magnet and the neutrophil fraction is eluted. The column is then washed with PBS (50 ml) and ethanol (absolute), and stored at 4° C.

Total cells are counted with a micro cell counter. One drop of lysogenic solution is added to the sample, which after 30s is recounted to assess contamination with erythrocytes. Cytospin smears are prepared on a Shandon Cytospin 2 cytospinner (100 μl samples, 3 minutes, 500 rpm). These preparations are stained and differential cell counts are determined by light microscopy, examining at least 500 cells. Cell viability is assessed by exclusion of trypan blue.

Eosinophils or neutrophils are diluted in HBSS and pipetted into 96 well microtiter plates (MTP) at 1-10×10³ cells/well. Each well contains a 200 μl sample comprising: 100 μl cell suspension; 50 μl HBSS; 10 μl lucigenin; 20 μl activation stimulus; and 20 μl test compound.

The samples are incubated with test compound or vehicle for 10 m prior to addition of an activation stimulus fMLP (1-10 μM) or C5a (1-100 nM) dissolved in dimethylsulfoxide and thereafter diluted in buffer, such that the highest solvent concentration used is 1% (at 100 μM test compound). MTPs are agitated to facilitate mixing of the cells and medium, and the MTP is placed into a luminometer. Total chemiluminescence and the temporal profile of each well is measured simultaneously over 20 m and the results expressed as arbitrary units, or as a percentage of fMLP-induced chemiluminescence in the absence of test compound. Results are fitted to the Hill equation and IC₅₀ values are calculated automatically.

The combinations of therapeutic agents of the present invention are active in the above test method at concentrations in the range of from 0.0001 μM to 0.5 μM, with preferred embodiments being active at concentrations in the range of from 0.1 nM to 100 nM.

The anti-inflammatory activity of the combinations of therapeutic agents of the present invention is additionally demonstrated by the inhibition of plasma extravasation into rat airways. In this assay tracheal tissue is taken and the extent of plasma leakage determined. This assay relates equally to other chronic inflammatory diseases of the airways including but not limited to COPD and accordingly is not recapitulated in that section.

Wistar albino rats (150-200 g) or Dunkin-Hartley guinea-pigs (450-600 g) are anaesthetised with sodium pentobarbitone and venous and arterial cannulae installed. Evans Blue dye to bind plasma proteins is administered i.v. (30 mg/kg). After 10 mins the test agents are administered i.t. and 10 mins later capsaicin administered i.v. (3ug/kg). 30 mins later, tracheal tissue is removed, extracted overnight into formamide and absorbance read at 620 nm. In some experiments the order of dosing was reversed such that the compounds were administered before the Evans Blue and inflammatory stimulus.

In the above test model In the above test model the combinations of therapeutic agents of the present invention exhibit anti-inflammatory activity at dosages in the range of from 0.001 to 0.1 mg/kg it.

From the above it may be seen that the combinations of therapeutic agents of the present invention are useful for the treatment of inflammatory or obstructive airways diseases or other conditions involving airways obstruction. In particular they are useful for the treatment of bronchial asthma.

In view of their anti-inflammatory activity and their influence on airways hyper-reactivity, the combinations of therapeutic agents of the present invention are useful for the treatment, in particular prophylactic treatment, of obstructive or inflammatory airways diseases. Thus, by continued and regular administration over prolonged periods of time the combinations of compounds of the present invention are useful in providing advance protection against the recurrence of bronchoconstriction or other symptomatic attack consequential to obstructive or inflammatory airways diseases. The combinations of compounds of the present invention are also useful for the control, amelioration or reversal of the basal status of such diseases.

Having regard to their bronchodilator activity the combinations of therapeutic agents of the present invention are useful as bronchodilators, e.g., in the treatment of chronic or acute bronchoconstriction, and for the symptomatic treatment of obstructive or inflammatory airways diseases.

Obstructive or inflammatory airways diseases to which the present invention applies include asthma; pneumoconiosis; chronic eosinophilic pneumonia; chronic obstructive airways or pulmonary disease (COAD or COPD); and adult respiratory distress syndrome (ARDS), as well as exacerbation of airways hyper-reactivity consequent to other drug therapy, e.g., aspirin or β-agonist therapy.

The selective adenosine A₂ₐ receptor agonists and anticholinergic compounds of the present invention can be administered alone or in combination but will generally be administered in admixture with a suitable pharmaceutical excipient, diluent or carrier.

The selective adenosine A₂ₐ receptor agonists and anticholinergic compounds of the present invention are preferably administered by inhalation and are conveniently delivered in the form of a dry powder (either alone or as a mixture, for example a mixture with lactose) from a dry powder inhaler or an aerosol spray presentation from a pressurised container, pump, spray, atomiser (preferably an atomiser using electrohydrodynamics to produce a fine mist) or nebuliser, with or without the use of a suitable propellant, e.g. dichlorodifluoromethane, trichlorofluoromethane, dichlorotetrafluoroethane, a hydrofluoroalkane such as 1,1,1,2-tetrafluoroethane (HFA 134A [trade mark]) or 1,1,1,2,3,3,3-heptafluoropropane (HFA 227EA [trade mark]), carbon dioxide, a further perfluorinated hydrocarbon such as Perflubron (trade mark) or other suitable gas. In the case of a pressurised aerosol, the dosage unit may be determined by providing a valve to deliver a metered amount. The pressurised container, pump, spray, atomiser or nebuliser may contain a solution or suspension of the active compound, e.g. using a mixture of ethanol (optionally, aqueous ethanol) or a suitable agent for dispersing, solubilising or extending release and the propellant as the solvent, which may additionally contain a lubricant, e.g. sorbitan trioleate. Capsules, blisters and cartridges (made, for example, from gelatin or HPMC) for use in an inhaler or insufflator may be formulated to contain a powder mix of the compound of the invention, a suitable powder base such as lactose or starch and a performance modifier such as I-leucine, mannitol or magnesium stearate.

Prior to use in a dry powder formulation or suspension formulation for inhalation the compound of the invention will be micronised to a size suitable for delivery by inhalation (typically considered as less than 5 microns). Micronisation could be achieved by a range of methods, for example spiral jet milling, fluid bed jet milling or use of supercritical fluid crystallisation.

A suitable solution formulation for use in an atomiser using electrohydrodynamics to produce a fine mist may contain from 1 μg to 10 mg of the compound of the invention per actuation and the actuation volume may vary from 1 to 100 μl. A typical formulation may comprise a compound of the invention, propylene glycol, sterile water, ethanol and sodium chloride. Alternative solvents may be used in place of propylene glycol, for example glycerol or polyethylene glycol.

Aerosol or dry powder formulations are preferably arranged so that each metered dose or “puff” contains from 1 to 4000 μg of a compound of the invention for delivery to the patient. The overall daily dose with an aerosol will be in the range of from 1 g to 20 mg which may be administered in a single dose or, more usually, in divided doses throughout the day.

The preferred ratio, by weight (w/w), of selective adenosine A₂ₐ receptor agonist:anticholinergic agent used will depend on the particular combination being examined. This is due to differences in the potency of individual compounds. The physician in any event will determine the actual dosage of each compound which will be most suitable for any individual patient and it will vary with the age, weight and response of the particular patient.

It is to be appreciated that all references herein to treatment include curative, palliative and prophylactic treatment.

1. An inhaled combination of a selective adenosine A₂ₐ receptor agonist and an anticholinergic agent, with the proviso that the anticholinergic agent is not a tiotropium salt.

2. A combination as claimed in claim 1 wherein the selective adenosine A₂ₐ receptor agonist is a compound generally or specifically disclosed in WO-A-00/23457, WO-A-00/77018, WO-A-01/27131, WO-A-01/27130, WO-A-01/60835, WO-A-02/00676 or WO-A-01/94368.

3. A combination as claimed in claim 2 wherein the selective adenosine A₂ₐ receptor agonist is:
N-({9-[(2R, 3R,4S,5R)-3,4-dihydroxy-5-(methoxymethyl)tetrahydro-2-furanyl]-6-[(2,2-diphenylethyl)amino]-9H-purin-2-yl}methyl)-2-methyl-1-propanesulfonamide (Example 15 of WO-A-00/23457);
cis -(2R,3R,4S,5R)-2-(6-[(2,2-diphenylethyl)amino]-2-{[(4-isopropylcyclohexyl)amino]methyl}-9H-purin-9-yl)-5-(methoxymethyl)tetrahydro-3,4-furandiol and trans-(2R,3R,4S,5R)-2-(6-[(2,2-diphenylethyl)amino]-2-{[(4-isopropylcyclohexyl)amino]methyl}-9H-purin-9-yl)-5-(methoxymethyl)tetrahydro-3,4-furandiol (Example 17 of WO-A-00/23457);
N-({9-[(2R,3R,4S,5R)-3,4-dihydroxy-5-(hydroxymethyl)tetrahydro-2-furanyl]-6-[(2,2-diphenylethyl)amino]-9H-purin-2-yl}methyl)-2-methyl-1-propanesulfonamide (Example 1 of WO-A-01/27130);
(2S,3S,4R,5R)-5-(6-[(2,2-diphenylethyl)amino]-2-{[(isopropylsulfonyl)amino]methyl}-9H-purin-9-yl)-N-ethyl-3,4-dihydroxytetrahydro-2-furancarboxamide (Example 3 of WO-A-01/27131);
9-[(2R,3R,4S,5R)-3,4-dihydroxy-5-(hydroxymethyl)tetrahydro-2-furanyl]-6-[(2,2-diphenylethyl)amino]-N-[2-(1-piperidinyl)ethyl]-9H-purine-2-carboxamide (Example 1 of WO-A-00/77018);
6-[(2,2-diphenylethyl)amino]-9-{(2R,3R,4S,5S)-5-[(ethylamino)carbonyl]-3,4-dihydroxytetrahydro-2-furanyl}-N-[2-(1-piperidinyl)ethyl]-9H-purine-2-carboxamide (Example 1 of WO-A-01/60835);
N-({9-[(2R,3R,4S,5R)-3,4-dihydroxy-5-(hydroxymethyl)tetrahydro-2-furanyl]-6-[(2,2-diphenylethyl)amino]-9H-purin-2-yl}methyl)-N′-[2-(diisopropylamino)ethyl]urea (Example 1 of WO-A-02/00676); or
6-[(2,2-diphenylethyl)amino]-9-{(2R,3R,4S,5S)-5-[(ethylamino)carbonyl]-3,4-dihydroxytetrahydro-2-furanyl}-N-{2-[({[1-(2-pyridinyl)-4-piperidinyl]amino}carbonyl)amino]ethyl}-9H-purine-2-carboxamide (Example 8 of WO-A-01/94368);
or a pharmaceutically acceptable salt or solvate thereof.

4. A combination as claimed in claim 3 wherein the selective adenosine A₂ₐ receptor agonist is 9-[(2R,3R,4S,5R)-3,4-dihydroxy-5-(hydroxymethyl)tetrahydro-2-furanyl]-6-[(2,2-diphenylethyl)amino]-N-[2-(1-piperidinyl)ethyl]-9H-purine-2-carboxamide or 6-[(2,2-diphenylethyl)amino]-9-{(2R,3R,4S,5S)-5-[(ethylamino)carbonyl]-3,4-dihydroxytetrahydro-2-furanyl}-N-{2-[({[1-(2-pyridinyl)-4-piperidinyl]amino}carbonyl)amino]ethyl}-9H-purine-2-carboxamide or a pharmaceutically acceptable salt or solvate thereof.

5. A combination as claimed in any one of the preceding claims wherein the anticholinergic agent is an ipratropium or an oxitropium salt or solvate thereof.

6. A combination as claimed in claim 1 wherein:
the adenosine A₂ₐ receptor agonist is 9-[(2R,3R,4S,5R)-3,4-dihydroxy-5-(hydroxymethyl)tetrahydro-2-furanyl]-6-[(2,2-diphenylethyl)amino]-N-[2-(1-piperidinyl)ethyl]-9H-purine-2-carboxamide or a pharmaceutically acceptable salt or solvate thereof and the anticholinergic agent is an ipratropium salt, or solvate thereof;
the adenosine A₂ₐ receptor agonist is 6-[(2,2-diphenylethyl)amino]-9-{(2R,3R,4S,5S)-5-[(ethylamino)carbonyl]-3,4-dihydroxytetrahydro-2-furanyl}-N-{2-[({[1-(2-pyridinyl)-4-piperidinyl]amino}carbonyl)amino]ethyl}-9H-purine-2-carboxamide or a pharmaceutically acceptable salt or solvate thereof and the anticholinergic agent is an ipratropium salt, or solvate thereof;
the adenosine A₂ₐ receptor agonist is 9-[(2R,3R,4S,5R)-3,4-dihydroxy-5-(hydroxymethyl)tetrahydro-2-furanyl]-6-[(2,2-diphenylethyl)amino]-N-[2-(1-piperidinyl)ethyl]-9H-purine-2-carboxamide or a pharmaceutically acceptable salt or solvate thereof and the anticholinergic agent is an oxitropium salt, or solvate thereof; or
the adenosine A₂ₐ receptor agonist is 6-[(2,2-diphenylethyl)amino]-9-{(2R,3R,4S,5S)-5-[(ethylamino)carbonyl]-3,4-dihydroxytetrahydro-2-furanyl}-N-{2-[({[1-(2-pyridinyl)-4-piperidinyl]amino}carbonyl)amino]ethyl}-9H-purine-2-carboxamide or a pharmaceutically acceptable salt or solvate thereof and the anticholinergic agent is an oxitropium salt, or solvate thereof.

7. A combination as claimed in any preceding claim for use as a medicament.

8. A combination as claimed in any one of claims 1 to 6 for simultaneous, sequential or separate administration in the treatment of an obstructive airways or other inflammatory disease.

9. A pharmaceutical composition comprising a selective adenosine A₂ₐ receptor agonist, an anticholinergic agent and a pharmaceutically acceptable excipient, diluent or carrier, for administration by the inhaled route in the treatment of an obstructive airways or other inflammatory disease, with the proviso that the anticholinergic agent is not a tiotropium salt.

10. A pharmaceutical composition, as claimed in claim 9, wherein the selective adenosine A₂ₐ receptor agonist and the anticholinergic agent are as defined in any one of claims 2 to 6.

11. The use of a selective adenosine A₂ₐ receptor agonist or an anticholinergic agent in the manufacture of a medicament for simultaneous, sequential or separate administration of both agents by the inhaled route in the treatment of an obstructive airways or other inflammatory disease, with the proviso that the anticholinergic agent is not a tiotropium salt.

12. The use as claimed in claim 11 wherein the selective adenosine A₂ₐ receptor agonist and the anticholinergic agent are as defined in any one of claims 2 to 6.

13. A method of treating of an obstructive airways or other inflammatory disease comprising administering simultaneously, sequentially or separately, by the inhaled route, to a mammal in need of such treatment, an effective amount of a selective adenosine A₂ₐ receptor agonist and an anticholinergic agent, with the proviso that the anticholinergic agent is not a tiotropium salt.

14. A method as claimed in claim 13 wherein the selective adenosine A₂ₐ receptor agonist and the anticholinergic agent are as defined in any one of claims 2 to 6.

15. An inhalation device for simultaneous, sequential or separate administration of a selective adenosine A₂ₐ receptor agonist and an anticholinergic agent in the treatment of an obstructive airways or other inflammatory disease, with the proviso that the anticholinergic agent is not a tiotropium salt.

16. A device as claimed in claim 15 wherein the selective adenosine A₂ₐ receptor agonist and the anticholinergic agent are as defined in any one of claims 2 to 6.
